(19) United States
(12) Patent Application Publication
(10) Pub. No.: US 2007/0280976 A1
(43) Pub. Date: Dec. 6, 2007

(54) Multi-phased personal care composition comprising a blooming perfume composition

(75) Inventors: Rebecca Ann Taylor, Cincinnati, OH (US); Karl Shiqing Wei, Mason, OH (US); Jimmie Lee Ward, Middletown, OH (US); Robert Richard Dykstra, West Chester, OH (US); Julie Anne Mathews, Okeana, OH (US); Lois Sara Gallon, Cincinnati, OH (US); Zerlina Guzdar Dubois, Mason, OH (US); Virginia Tzung-Hwei Hutchins, Cincinnati, OH (US); Jianjun Justin Li, West Chester, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/447,491

(22) Filed: Jun. 6, 2006

(51) Int. Cl. A61K 8/37; A61K 8/42

(52) U.S. Cl. 424/401; 512/1; 424/70.31

(57) ABSTRACT

A multi-phase personal care composition is described comprising is a first phase and a second phase. The personal care composition comprises at least 0.25%, by weight of the composition, of blooming perfume ingredients having a KI of less than about 1500.

### FIELD OF THE INVENTION

The present invention relates to a structured personal care composition comprising a perfume composition.

### BACKGROUND OF THE INVENTION

Personal care compositions are well known and widely used. Desirable personal care composition must meet a number of criteria. For example, in order to be acceptable to consumers, a personal care composition must exhibit good cleaning properties, must exhibit good lathering characteristics, must be mild to the skin (not cause drying or irritation) and preferably should even provide a conditioning benefit to the skin. Moreover, odor or scent is a product characteristic which drives consumer acceptance. Some consumers choose a personal care composition for both the odor of the product itself, as well as, the residual odor the composition leaves on the skin or hair. The product odor is the scent of the product in the bottle and the “bloom” or scent during use in the shower or bath. The residual odor is the scent of the product on the consumer's skin. Both are important to the consumers of personal care compositions.

Personal care compositions are known and widely used that have a product scent and leave a residual odor of the composition on the skin or hair. However, some consumers apply after shower products such as, aftershave, colognes, cologne spray, perfumed lotions, or fine fragrances to intentionally leave a distinct residual scent on their skin and hair. Because both the personal care compositions and after shower product have scents, consumer prefer personal care compositions that exactly match, compliment or are not stronger than the after shower products. However, some personal care compositions have strong residual scents that are very different from or stronger than a consumer's after shower product. These strong residual odors from the personal care composition sometime leave the consumer with more than one scent on their hair and skin which is not preferred.

One solution to this problem would be to not scent the personal care composition. However, many of the components of a personal care composition have base odor that would be unpleasant to the consumer if no scent was added. Moreover, consumers enjoy the scent of the personal care composition in the shower. Thus, there is a need for a personal cleansing composition that has a “bloom” or scent in the shower that leaves little to no residual odor on the skin and hair after the shower.

### SUMMARY OF THE INVENTION

The multi-phase personal care composition comprises a first phase and a second phase. The personal care composition comprises at least 0.25%, by weight of said multi-phase personal care composition of blooming perfume ingredients having a KI of less than about 1500.

The blooming perfume compositions comprised of ingredients having a KI of less than about 1500, as disclosed herein, can be formulated into personal care compositions and provide a significantly noticeable scent in the shower to the consumer while leaving little to no residual perfume of the skin and hair.

### DETAILED DESCRIPTION OF THE INVENTION

The term “ambient conditions” as used herein, refers to surrounding conditions at one (1) atmosphere of pressure, 50% relative humidity, and 25° C.

“Kovat's Index” (KI, or Retention Index) is defined by the selective retention of solutes or perfume raw materials (PRMs) onto a chromatographic column. It is primarily determined by the column stationary phase and the properties of solutes or PRMs. For a given column system, a PRM's polarity, molecular weight, vapor pressure, boiling point and the stationary phase property determine the extent of retention. To systematically express the retention of an analyte on a given GC column, a measure called Kovat's Index (or retention index) is defined. Kovat's Index (KI) places the volatility attributes of an analyte (or PRM) on a column in relation to the volatility characteristics of n-alkane series on that column. Typical columns used are DB-5 and DB-1.

By this definition the KI of a normal alkane is set to 100 n, where n=number of carbons atoms of the n-alkane. With this definition, the Kovat's index of a PRM, x, eluting at time t′, between two neighboring n-alkanes with number of carbon atoms n and N having corrected retention times t′ₙ and t′_{N} respectively will then be calculated as:

$$KI = 100 ( n + \frac{log {t}_{x}^{′} - log {t}_{n}^{′}}{log {t}_{N}^{′} - log {t}_{n}^{′}} ) \qquad ( 1 )$$

By the term “multi-phase” or “multi-phase” as used herein, is meant that the phases of the present compositions occupy separate but distinct physical spaces inside the package in which they are stored, but are in direct contact with one another (i.e., they are not separated by a barrier and they are not emulsified or mixed to any significant degree). In one preferred embodiment of the present invention, the “multi-phase” personal care compositions can comprise at least two visually distinct phases which are present within the container as a visually distinct pattern. The pattern results from the combination of the “multi-phase” composition by a process herein described. The “patterns” or “patterned” include but are not limited to the following examples: striped, marbled, rectilinear, interrupted striped, check, mottled, veined, clustered, speckled, geometric, spotted, ribbons, helical, swirl, arrayed, variegated, textured, grooved, ridged, waved, sinusoidal, spiral, twisted, curved, cycle, streaks, striated, contoured, anisotropic, laced, weave or woven, basket weave, spotted, and tessellated. Preferably the pattern is selected from the group consisting of striped, geometric, marbled, and combinations thereof. The phases may be various different colors, and/or include particles, glitter or pearlescent agents in at least one of the phases in order to offset its appearance from the other phase(s) present.

The term “multi-phase personal care composition” as used herein, refers to compositions intended for topical application to the skin or hair. Non-limiting examples of personal care compositions include skin care lotions, in-shower body moisturizers, body washes, bar soaps, shampoos, and conditioners.

The term “structured,” as used herein means having a rheology that confers stability on the multi-phase composition. The degree of structure is determined by the Yield Stress and Zero Shear Viscosity Method and by the Ultracentrifugation Method, both described hereafter. When a phase is a structured phase, typically it has a Yield Stress of greater than about 0.1 Pascal (Pa), more preferably greater than about 0.5 Pa, even more preferably greater than about 1.0 Pa, still more preferably greater than about 2.0 Pa, still even more preferably greater than about 3 Pa, and even still even more preferably greater than about 5 Pa as measured by the Yield Stress and Zero Shear Viscosity Method described hereafter. When a phase is a structured phase, it may also typically have a Zero Shear Viscosity of at least about 500 Pascal-seconds (Pa-s), preferably at least about 1,000 Pa-s, more preferably at least about 1,500 Pa-s, even more preferably at least about 2,000 Pa-s. Accordingly, when a cleansing phase or a surfactant phase of the multi-phase composition of the present invention is structured, it has a Structured Domain Volume Ratio as measured by the Ultracentrifugation Method described hereafter, of greater than about 40%, preferably at least about 45%, more preferably at least about 50%, more preferably at least about 55%, more preferably at least about 60%, more preferably at least about 65%, more preferably at least about 70%, more preferably at least about 75%, more preferably at least about 80%, even more preferably at least about 85%.

The term “surfactant component” as used herein means the total of all anionic, nonionic, amphoteric, zwitterionic and cationic surfactants in a phase. When calculations are based on the surfactant component, water and electrolyte are excluded from the calculations involving the surfactant component, since surfactants as manufactured typically are diluted and neutralized.

The term “visually distinct phase” as used herein, refers to a region of the multi-phase personal care composition having one average composition, as distinct from another region having a different average composition, wherein the regions are visible to the unaided naked eye. This would not preclude the distinct regions from comprising two similar phases where one phase could comprise pigments, dyes, particles, and various optional ingredients, hence a region of a different average composition. A phase generally occupies a space or spaces having dimensions larger than the colloidal or sub-colloidal components it comprises. A phase may also be constituted or re-constituted, collected, or separated into a bulk phase in order to observe its properties, e.g., by centrifugation, filtration or the like.

The multi-phase personal care composition comprises a first phase and a second phase. The first phase comprises at least 0.25%, by weight of the composition, of blooming perfume ingredients having a Kovat's Index of less than about 1500.

The multi-phase personal care composition of the present invention is typically extrudable or dispensible from a package. The multi-phase personal care compositions typically exhibit a viscosity of from about 1,500 centipoise (cP) to about 1,000,000 cP, as measured by the Viscosity Method as described in copending application Ser. No. 10/841174 filed on May 7, 2004 titled “Multi-phase Personal Care Compositions.”

When evaluating a multi-phase personal care composition, by the methods described herein, preferably each individual phase is evaluated prior to combining, unless otherwise indicated in the individual methodology. However, if the phases are combined, each phase can be separated by centrifugation, ultracentrifugation, pipetting, filtering, washing, dilution, concentration, or combination thereof, and then the separate components or phases can be evaluated. Preferably, the separation means is chosen so that the resulting separated components being evaluated is not destroyed, but is representative of the component as it exists in the multi-phase personal care composition, i.e., its composition and distribution of components therein is not substantially altered by the separation means. Generally, multi-phase compositions comprise domains significantly larger than colloidal dimensions so that separation of the phases into the bulk is relatively easy to accomplish while retaining the colloidal or microscopic distribution of components therein. Preferably, the compositions of the present invention are rinse-off formulations, by which is meant the product is applied topically to the skin or hair and then subsequently (i.e., within minutes) the skin or hair is rinsed with water, or otherwise wiped off using a substrate or other suitable removal means with deposition of a portion of the composition.

The multi-phase personal care compositions of the present invention can comprise at least two visually distinct phases, wherein the composition can have a first structured phase, a second phase, a third phase, a fourth phase and so on. The ratio of a first phase to a second phase is preferably from about 1:99 to about 99:1, preferably from about 90:10 to about 10:90, more preferably from about 80:20 to about 20:80, even more preferably from about 70:30 to about 30:70, still even more preferably from about 60:40 to about 40:60, even still even more preferably about 50:50. The preferred pH range of the multi-phase personal care composition is from about 5 to about 8. Each phase could be one or more of the following nonlimiting examples including: a cleansing phase, a benefit phase, and a non-lathering structured aqueous phase, which are described in greater detail hereinafter.

The multi-phase composition comprises at least 0.25%, by weight of said personal care composition, of blooming perfume ingredients having a KI of less than about 1500. In some embodiments, the multi-phase composition comprises at least 0.35%, by weight of said personal care composition, of blooming perfume ingredients having a KI of less than about 1500. In other embodiments, the multi-phase composition comprises at least 0.40%, by weight of said personal care composition, of blooming perfume ingredients having a KI of less than about 1500. The blooming perfume ingredients have a boiling point of less than about 260° C., a ClopP of from about 1.5 to about 4.0 preferably from about 2.0 to about 4.0, more preferably 2.3 from about to about 4.0, most preferably from about 2.5 to about 4.0. Examples of blooming ingredients are illustrated in Table 1.

TABLE 1
Blooming Perfume Ingredients

| Blooming | | Kovat | Boiling | |
|---|---|---|---|---|
| ingredients | INCI Name | Index | Point | ClogP |
| Beta Gamma | 2-Hexen-1-ol | 870 | 159.6 ± 8.0 | 1.755 ± 0.212 |
| Hexenol | | | | |
| Cis 3 | (Z)-3-Hexen-1-ol | 1006 | 174.2 ± 19.0 | 2.508 ± 0.222 |
| Hexenyl | acetate | | | |
| Acetate | | | | |
| Cyclo | Cyclo Galbanate | 1434 | 283.1 ± 15.0 | 2.975 ± 0.341 |
| Galbanate | | | | |
| Dihydro | 2,6-dimethyl-7- | 1074 | 188.4 ± 0.0 | 3.004 ± 0.222 |
| Myrcenol | Octen-2-ol | | | |
| Ethyl | Ethyl Caproate | 1002 | 167.9 ± 3.0 | 2.834 ± 0.205 |
| Caproate | | | | |
| Ethyl-2- | Butanoic acid, 2- | 848 | 135.1 ± 8.0 | 2.118 ± 0.212 |
| methyl | methyl-, ethyl | | | |
| Butyrate | ester | | | |
| Hexyl | Acetic acid, hexyl | 1012 | 171.5 ± 3.0 | 2.834 ± 0.205 |
| Acetate | ester | | | |
| Melonal | 2,6-Dimethyl-5- | 1058 | 187.7 ± 19.0 | 3.003 ± 0.261 |
| | heptenal | | | |
| Triplal | 2,4-Dimethyl-3- | 1091 | 189.2 ± 20.0 | 2.670 ± 0.245 |
| | cyclohexene-1- | | | |
| | carboxaldehyde | | | |
| Anethol Usp | Benzene, | 1310 | 237.5 ± 9.0 | 3.168 ± 0.217 |
| | 1-methoxy- | | | |
| | 4-(1-propenyl)- | | | |
| Gamma | 2(3H)-Furanone, | 1485 | 266.7 ± 8.0 | 2.385 ± 0.278 |
| Decalactone | 5-hexyldihydro- | | | |
| Hydroxycitro | Octanal, | 1292 | 251.6 ± 23.0 | 1.539 ± 0.244 |
| nellal | 7-hydroxy- | | | |
| | 3,7-dimethyl- | | | |
| Decyl | Decanal | 1209 | 209.0 ± 3.0 | 4.094 ± 0.223 |
| Aldehyde | | | | |

The multi-phase, personal care composition comprising comprises a blooming perfume composition comprising preferably at least 20% by weight of the blooming perfume composition, more preferably at least 30% by weight of the blooming perfume composition, more preferably at least 50% by weight of the blooming perfume composition, more preferably at least 70% by weight of the blooming perfume composition, more preferably least 80% by weight of the blooming perfume composition, most preferably least 90% by weight of the blooming perfume composition, of blooming perfume ingredients KI of less than about 1500.

A blooming perfume ingredient is characterized by its boiling point (B.P.) and its octanol/water partition coefficient (P). The octanol/water partition coefficient of a perfume ingredient is the ratio between its equilibrium concentrations in octanol and in water. The preferred perfume ingredients of this invention have a B.P., determined at the normal, standard pressure of about 760 mm Hg, of about 260° C. or lower, preferably less than about 255° C.; and more preferably less than about 250° C., and an octanol/water partition coefficient P of about 1,000 or higher. Since the partition coefficients of the preferred perfume ingredients of this invention have high values, they are more conveniently given in the form of their logarithm to the base 10, logP. Thus the preferred perfume ingredients of this invention have ClogP at 25° C. of about 1.5 to about 4.0, preferably from about 2.0 to about 4.0, more preferably from about 2.3 to about 4.0, and most preferably 2.5-4.0.

The boiling points of many perfume compounds can be found using the SciFinder (http://scifinder.cas.org/). When unreported, the 760 mm boiling points of perfume ingredients can be obtained through SciFinder where the calculated values of boiling point using Advanced Chemistry Development (ACD/Labs) Software Solaris V4.67 are listed. The ACD/Labs calculated boiling point values, which are the most reliable and widely used estimates for this property, are preferably used instead of the experimental boiling point values in the selection of perfume ingredients which are useful in the present invention.

The logP of many perfume ingredients has been reported; for example, the Pomona92 database, available from Daylight Chemical Information Systems, Inc. (Daylight CIS), Irvine, Calif., contains many, along with citations to the original literature. However, the logP values are most conveniently obtained through SciFinder where the calculated values of log P using Advanced Chemistry Development (ACD/Labs) Software Solaris V4.67 are listed. The ClogP values, which are the most reliable and widely used estimates for this physicochemical property, are preferably used instead of the experimental logP values in the selection of perfume ingredients which are useful in the present invention. The ClogP values were obtained through SciFinder where the calculated values of log P using Advanced Chemistry Development (ACD/Labs) Software Solaris V4.67 are listed.

Thus, when a perfume composition which is composed of ingredients having a B.P. of about 260° C. or lower and a ClogP, or an experimental logP, of from about 1.5 to about 4.0, is used in the shower or bath, the perfume is very effusive and very noticeable when the product is used.

The blooming perfume compositions of the present invention contain at least 5, preferably at least 6, more preferably at least 7, even more preferably at least 8 or 9 or even 10 or more different blooming perfume ingredients.

Most common perfume ingredients which are derived from natural sources are composed of a multitude of components. For example, orange terpenes contain about 90% to about 95% d-limonene, but also contain many other minor ingredients. When each such material is used in the formulation of blooming perfume compositions of the present invention, it is counted as one ingredient, for the purpose of defining the invention. Synthetic reproductions of such natural perfume ingredients are also comprised of a multitude of components and are counted as one ingredient for the purpose of defining the invention.

The blooming perfume ingredients have a gas chromatographic Kovat's Index (as determined on 5% phenyl-methylpolysiloxane as non-polar silicone stationary phase) of less than 1500.

The blooming perfume composition of the present invention can optionally contain “non-blooming” perfume ingredients. The optional non-blooming perfume ingredients of this invention have a KI value greater than 1500, a boiling point measured at the normal, standard pressure, of about 260° C. or higher, and a ClogP of greater than about 2.5. Thus, when a perfume composition is composed of some preferred blooming ingredients and some non-blooming ingredients, the perfume effect is longer lasting when the product is used. Non-blooming perfume ingredients are used primarily in applications where the water will evaporate, thus liberating the perfume. Table 2 illustrates examples of non-blooming ingredients.

TABLE 2
Non-blooming Ingredients

| Non- | | | | |
|---|---|---|---|---|
| blooming | | Kovat | Boiling | |
| Ingredients | INCI Name | Index | Point | ClogP |
| Sanjinol | 2-Buten-1-ol, 2-ethyl-4- | 1582 | 287.4 ± 9.0 | 4.965 ± 0.274 |
| | (2,2,3-trimethyl-3- | | | |
| | cyclopenten-1-yl)- | | | |
| Polysantol | 4-Penten-2-ol, 3,3- | 1517 | 299.7 ± 9.0 | 4.778 ± 0.263 |
| | dimethyl-5-(2,2,3- | | | |
| | trimethyl-3-cyclopenten-1-yl)- | | | |
| Lyral | 3-Cyclohexene-1- | 1687 | 318.7 ± 27.0 | 2.532 ± 0.257 |
| | carboxaldehyde, 4-(4- | | | |
| | hydroxy-4-methylpentyl)- | | | |
| Ambrettolide | Oxacycloheptandec-10-en- | 2005 | 399.2 ± 27.0 | 5.516 ± 0.287 |
| | 2-one | | | |
| Hexyl | Octanal, 2- | 1772 | 308.1 ± 0.0 | 5.332 ± 0.374 |
| Cinnamic | (phenylmethylene)- | | | |
| Aldehyde | | | | |
| Delta | 3- | 1917 | 329.5 ± 10.0 | 6.333 ± 0.255 |
| Muscenone | Methylcyclopentadecenone | | | |
| Ionone | 3-Buten-2-one, 3-methyl- | 1502 | 285.30 ± 20.0 | 4.409 ± 0.272 |
| Gamma | 4-(2,6,6-trimethyl-2- | | | |
| Methyl | cyclohexen-1-yl)- | | | |
| Iso E Super | 7-acetyl,1,2,3,4,5,6,7,8 - | 1699 | 312.2 ± 22.0 | 5.285 ± 0.223 |
| | octahydro-1,1,6,7- | | | |
| | tetramethyl naphthalene | | | |
| Methyl | Cyclopentaneacetic acid, 3- | 1670 | 307.8 ± 15.0 | 2.496 ± 0.274 |
| dihydrojasm | oxo-2-pentyl-, methyl ester | | | |
| onate | | | | |
| Phenoxy | Propanoic acid, 2-methyl-, | 1528 | 273.800 ± 13.0 | 2.973 ± 0.248 |
| Ethyl Iso | 2-phenoxyethyl ester | | | |
| Butyrate | | | | |

The multiphase composition comprises a total perfume composition is comprised of the blooming perfume ingredients and the non-blooming perfume ingredients (sum of blooming and non-blooming). When non-blooming perfume ingredients are used in combination with the blooming perfume ingredients in the blooming perfume compositions of the present invention, the weight percentage of blooming perfume ingredients is typically at least 10% by weight of the total perfume composition, at least about 20% by weight of the total perfume composition, preferably at least about 50% by weight of the total perfume composition and more preferably at 100% by weight of the total perfume composition.

In the perfume art, some auxiliary materials having no odor, or a low odor, are used, e.g., as solvents, diluents, extenders or fixatives. Non-limiting examples of these materials are ethyl alcohol, carbitol, dipropylene glycol, diethyl phthalate, triethyl citrate, isopropyl myristate, and benzyl benzoate. These materials are used for, e. g., solubilizing or diluting some solid or viscous perfume ingredients to, e. g., improve handling and/or formulating. These materials are useful in the blooming perfume compositions, but are not counted in the calculation of the limits for the definition/formulation of the blooming perfume compositions of the present invention.

The non-blooming perfume ingredients of present invention also comprise from about 0% to about 80%, preferably from about 10% to about 50%, more preferably from about 20% to about 40%, and most preferably from about 25% to about 35%, of non-blooming perfume ingredients having a KI value greater than 1500, a B.P. of more than about 260° C. and having a ClogP of greater than about 2.5. In certain personal care composition, some non-blooming perfume ingredients can be used in small amounts, e.g., to improve overall perfume odor. These ingredients are particularly effective at masking base odors from surfactants and/or other detergent ingredients. When used at the low levels herein, an improved blooming perfume composition is obtained that betters masks base odors while still minimizing residual perfume on skin and hair.

The first phase or second phase of the multi-phase personal care composition of the present invention can be a cleansing phase. Preferably, the surfactant component comprises a mixture of surfactants. The multi-phase personal care composition typically comprises from about 1% to about 99%, by weight of the composition, of said cleansing phase.

The surfactant component preferably comprises a lathering surfactant or a mixture of lathering surfactants. The surfactant component comprises surfactants suitable for application to the skin or hair. Suitable surfactants for use herein include any known or otherwise effective cleansing surfactant suitable for application to the skin, and which are otherwise compatible with the other essential ingredients in the multi-phase personal care composition including water. These surfactants include anionic, nonionic, cationic, zwitterionic, amphoteric surfactants, soap, or combinations thereof. Preferably, anionic surfactant comprises at least 40% of the surfactant component, more preferably from about 45% to about 95% of the surfactant component, even more preferably from about 50% to about 90%, still more preferably from about 55% to about 85%, and even still most preferably at least about 60% of the surfactant component comprises anionic surfactant.

The multi-phase personal care composition preferably comprises a surfactant component at concentrations ranging from about 2% to about 40%, more preferably from about 3% to about 30%, even more preferably from about 4% to about 25%, still more preferably from about 5% to about 20%, still even more preferably from about 10% to about 20%, and even still even more preferably from about 15% to about 20%, by weight of the first phase.

The surfactant component is preferably a structured domain comprising surfactants. The structured domain enables the incorporation of high levels of benefit components in a separate phase that are not emulsified in the composition. In a preferred embodiment the structured domain is an opaque structured domain. The opaque structured domain is preferably a lamellar phase. The lamellar phase produces a lamellar gel network. The lamellar phase can provide resistance to shear, adequate yield to suspend particles and droplets and at the same time provides long term stability, since it is thermodynamically stable. The lamellar phase tends to have a higher viscosity thus minimizing the need for viscosity modifiers.

The multi-phase, personal care composition typically provides a Total Lather Volume of at least about 600 ml, preferably greater than about 800 ml, more preferably greater than about 1000 ml, even more preferably greater than about 1200 ml, and still more preferably greater than about 1500 ml, as measured by the Lather Volume Test described hereafter. The multi-phase, personal care composition preferably has a Flash Lather Volume of at least about 300 ml, preferably greater than about 400 ml, even more preferably greater than about 500 ml, as measured by the Lather Volume Test described hereafter.

Suitable surfactants are described in McCutcheon's, Detergents and Emulsifiers, North American edition (1986), published by allured Publishing Corporation; and McCutcheon's, Functional Materials, North American Edition (1992); and in U.S. Pat. No. 3,929,678 issued to Laughlin, et al on Dec. 30, 1975.

Preferred linear anionic surfactants for use in the surfactant component of the multi-phase, personal care composition include ammonium lauryl sulfate, ammonium laureth sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, potassium lauryl sulfate, and combinations thereof.

Branched anioinc surfactants and monomethyl branched anionic surfactants suitable for the present invention are described in commonly owned U.S. Application Ser. No. 60/680,149 entitled “Structured Multi-phased Personal Cleansing Compositions Comprising Branched Anionic Surfactants” filed on May 12, 2004 by Smith, et al. Branched anionic surfactants include but are not limited to the following surfactants: sodium trideceth sulfate, sodium tridecyl sulfate, sodium C₁₂₋₁₃ alkyl sulfate, and C₁₂₋₁₃ pareth sulfate and sodium C₁₂₋₁₃ pareth-n sulfate. Branched surfactants can be derived from synthetic alcohols such as the primary alcohols from the liquid hydrocarbons produced by Fischer-Tropsch condensed syngas, for example Safol™ 23 Alcohol available from Sasol North America, Houston, Tex.; from synthetic alcohols such as Neodol™ 23 Alcohol available from Shell Chemicals, USA; from synthetically made alcohols such as those described in U.S. Pat. No. 6,335,312 issued to Coffindaffer, et al on Jan. 1, 2002. Sulfates can be prepared by conventional processes to high purity from a sulfur based SO₃ air stream process, chlorosulfonic acid process, sulfuric acid process, or Oleum process. Preparation via SO₃ air stream in a falling film reactor is a preferred sulfation process.

Amphoteric surfactants suitable for use in the multi-phase, personal care composition include those that are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition are sodium 3-dodecyl-aminopropionate, sodium 3-dodecylaminopropane sulfonate, sodium lauryl sarcosinate, and N-alkyltaurines such as the one prepared by reacting dodecylamine with sodium isethionate according to the teaching of U.S. Pat. No. 2,658,072 issued to Kosmin, et al. Amphoacetates and diamphoacetates, may also be used. Sodium lauroamphoacetate, sodium cocoamphoactetate, disodium lauroamphoacetate, and disodium cocodiamphoacetate are preferred in some embodiments.

Zwitterionic surfactants suitable for use in the multi-phase, personal care composition include those that are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. Other zwitterionic surfactants suitable for use in the multi-phase, personal care composition include betaines, including high alkyl betaines such as, coco dimethyl carboxymethyl betaine, cocoamidopropyl betaine, cocobetaine. and carboxymethyl betaine.

Non-limiting examples of preferred nonionic surfactants for use herein are those selected form the group consisting of C₈-C₁₄ glucose amides, C₈-C₁₄ alkyl polyglucosides, sucrose cocoate, sucrose laurate, alkanolamides, ethoxylated alcohols and mixtures thereof. In a preferred embodiment the nonionic surfactant is selected from the group consisting of glyceryl monohydroxystearate, steareth-2, isosteareth-2, hydroxy stearic acid, propylene glycol stearate, PEG-2 stearate, sorbitan monostearate, glyceryl stearate, glyceryl laurate, laureth-2, cocamide monoethanolamine, lauramide monoethanolamine, and mixtures thereof.

Mixtures of anionic surfactants may be used in some embodiments, including mixtures of linear and branched surfactants, and anionic surfactants with nonionic, amphoteric, and/or zwitterionic surfactants.

The electrolyte, if used, can be added per se to the multi-phase personal care composition or it can be formed in situ via the counterions included in one of the raw materials. The electrolyte preferably includes an anion comprising phosphate, chloride, sulfate or citrate and a cation comprising sodium, ammonium, potassium, magnesium or mixtures thereof. Some preferred electrolytes are sodium chloride, ammonium chloride, sodium or ammonium sulfate. The electrolyte is preferably added to the surfactant component of the composition in the amount of from about 0.1% to about 15% by weight, preferably from about 1% to about 6% by weight of the multi-phase personal care composition, but may be varied if required.

In one embodiment of the present invention, the multi-phase, personal care composition comprises a surfactant component comprising a mixture of at least one nonionic surfactant, at least one anionic surfactant and at least one amphoteric surfactant, and an electrolyte. In another one embodiment, the surfactant can comprise a mixtures of surfactants, water, at least one anionic surfactant, an electrolyte, and at least one alkanolamide. The amount of alkanolamide in the composition is typically from about 0.1% to about 10%, preferably from about 2% to about 5%, by weight of the cleansing phase.

The first phase or second phase of the multi-phase, personal care compositions of the present invention can be a benefit phase. The benefit phase in the present invention is preferably anhydrous. The benefit phase typically comprises hydrophobic materials. The benefit phase comprises from about 1% to about 100%, preferably at least about 35%, most preferably at least about 50%, by weight of the benefit phase, of a hydrophobic material. The hydrophobic materials suitable for use in the present invention preferably have a Vaughan Solubility Parameter of from about 5 to about 15 (cal/cm³)^{1/2}, as defined by Vaughan in Cosmetics and Toiletries, Vol. 103. Non-limiting examples of hydrophobic materials having VSP values ranging from about 5 to about 15 include the following: Cyclomethicone 5.92, Squalene 6.03, Petrolatum 7.33, Isopropyl Palmitate 7.78, Isopropyl Myristate 8.02, Castor Oil 8.90, Cholesterol 9.55, as reported in Solubility, Effects in Product, Package, Penetration and Preservation, C. D. Vaughan, Cosmetics and Toiletries, Vol. 103, October 1988.

The hydrophobic compositions are preferably selected among those having defined rheological properties as described hereinafter, including selected Consistency value (K) and Shear Index (n). These preferred rheological properties are especially useful in providing the multi-phase, personal care compositions with improved deposition of hydrophobic materials. The benefit phase has a Consistency Value (K) from about 20 to about 2,000 Pa-s, preferably from about 25 to about 500 Pa-s, more preferably from about 30 to about 450 Pa-s, still more preferably from about 30 to about 400 Pa-s and even still more preferably from about 30 to about 350 Pa-s. The benefit phase has a Shear Index from about 0.025 to about 0.99, preferably from about 0.05 to about 0.70 and more preferably from about 0.09 to about 0.60.

Nonlimiting examples of hydrophobic material suitable for use herein can include a variety of hydrocarbons, oils and waxes, silicones, fatty acid derivatives, cholesterol, cholesterol derivatives, diglycerides, triglycerides, vegetable oils, vegetable oil derivatives, acetoglyceride esters, alkyl esters, alkenyl esters, polyglycerin fatty acid esters, lanolin and its derivatives, wax esters, beeswax derivatives, sterols and phospholipids, and combinations thereof.

The benefit phase of the composition preferably can comprise one or more hydrophobic materials, wherein at least 1% by weight of the hydrophobic materials are selected from petrolatum, mineral oil, sunflower seed oil, alkyl siloxanes, polymethylsiloxanes and methylphenylpolysiloxanes, and combinations thereof. More preferably, at least about 20% by weight of the hydrophobic materials are selected from the groups of petrolatum, mineral oil, paraffins, polyethylene, polydecene, dimethicones, alkyl siloxanes, lanolins. More preferably, at least about 50% by weight of the hydrophobic materials are selected from the groups of petrolatum, mineral oil, paraffins, polyethylene, polydecene, dimethicones, alkyl siloxanes, lanolins.

Examples of suitable benefit phases and description of measuring the values of Consistency (K) and Shear Index (n) are described in U.S. patent application Ser. No. 10/665,670, Publication No. 2004/0057920 A1 entitled Striped liquid personal cleansing compositions containing a cleansing phase and a separate benefit phase” filed by Fact, et al. on Sep. 18, 2003, published on Apr. 4, 2004, U.S. patent application Ser. No. 10/699,469 Publication No. 2004/0092415 A1 entitled “Striped liquid personal cleansing compositions containing a cleansing phase and a separate benefit phase with improved stability” filed by Fact, et al. on Oct. 31, 2003, published on May 13, 2004 and U.S. patent application Ser. No. 10/837,214 Publication No. 2004/0219119 A1 entitled “Visually distinctive multiple liquid phase compositions” filed by Weir, et al. on Apr. 30, 2004, published on Nov. 18, 2004.

The first phase or second phase of the multi-phase personal care compositions of the present invention can comprise a structured aqueous phase that comprises a water structurant and water. The structured aqueous phase can be hydrophilic and in a preferred embodiment the structured aqueous phase is a hydrophilic, non-lathering gelled water phase. In addition, the structured aqueous phase typically comprises less than about 5%, preferably less than about 3%, and more preferably less than about 1%, by weight of the structured aqueous phase, of a surfactant. In one embodiment of the present invention, the structured aqueous phase is free of lathering surfactant in the formulation.

The structured aqueous phase of the present invention can comprise from about 30% to about 99%, by weight of the structured aqueous phase, of water. The structured aqueous phase generally comprises more than about 50%, preferably more than about 60%, even more preferably more than about 70%, still more preferably more than about 80%, by weight of the structured aqueous phase, of water.

The structured aqueous phase will typically have a pH of from about 5 to about 9.5, more preferably about 7. A water structurant for the structured aqueous phase can have a net cationic charge, net anionic charge, or neutral charge. The structured aqueous phase of the present compositions can further comprise optional ingredients such as, pigments, pH regulators (e.g. triethanolamine), and preservatives.

The structured aqueous phase can comprise from about 0.1% to about 30%, preferably from about 0.5% to about 20%, more preferably from about 0.5% to about 10%, and even more preferably from about 0.5% to about 5%, by weight of the structured aqueous phase, of a water structurant.

The water structurant is typically selected from the group consisting of inorganic water structurants, charged polymeric water structurants, water soluble polymeric structurants, associative water structurants, and mixtures thereof. Non-limiting examples of inorganic water structurants include silicas, polymeric gellants such as polyacrylates, polyacrylamides, starches, modified starches, crosslinked polymeric gellants, copolymers, and mixtures thereof. Non-limiting examples of charged polymeric water structurants for use in the multi-phase personal care composition include Acrylates/Vinyl Isodecanoate Crosspolymer (Stabylen 30 from 3V), Acrylates/C10-30 Alkyl Acrylate Crosspolymer (Pemulen TR1 and TR2), Carbomers, Ammonium Acryloyidimethyltaurate/VP Copolymer (Aristoflex AVC from Clariant), Ammonium Acryloyidimethyltaurate/Beheneth-25 Methacrylate Crosspolymer (Aristoflex HMB from Clariant), Acrylates/Ceteth-20 Itaconate Copolymer (Structure 3001 from National Starch), Polyacrylamide (Sepigel 305 from SBPPIC), and mixtures thereof. Non-limiting examples of water soluble polymeric structurants for use in the multi-phase personal care composition include cellulose gums and gel, and starches. Non-limiting examples of associative water structurants for use in the multi-phase personal care composition include xanthum gum, gellum gum, pectins, alginates such as propylene glycol alginate, and mixtures thereof.

The phases of the multi-phase personal care composition, preferably the cleansing phase, can further comprise a polymeric phase structurant. The compositions of the present invention typically can comprise from about 0.05% to about 10%, preferably from about 0.1% to about 4% and more preferably from about 0.2% to about 2% by weight of the phase, of a polymeric phase structurant. Non-limiting examples of polymeric phase structurant include but is not limited to the following examples: deflocculating polymers, naturally derived polymers, synthetic polymers, crosslinked polymers, block polymers, block copolymers, copolymers, hydrophilic polymers, nonionic polymers, anionic polymers, hydrophobic polymers, hydrophobically modified polymers, associative polymers, oligomers, and copolymers thereof as described in U.S Patent Application No. 60/628,036 filed on Nov. 15, 2003 by Wagner, et al titled “Depositable Solids.” Preferably the polymeric phase structurant can be crosslinked. These polymeric phase structurant useful in the present invention are more fully described in U.S. Pat. No. 5,087,445, to Haffey et al., issued Feb. 11, 1992; U.S. Pat. No. 4,509,949, to Huang et al., issued Apr. 5, 1985, U.S. Pat. No. 2,798,053, to Brown, issued Jul. 2, 1957. See also, CTFA International Cosmetic Ingredient Dictionary, fourth edition, 1991, pp. 12 and 80.

The phase of the present compositions, preferably the cleansing phase, optionally can further comprise a liquid crystalline phase inducing structurant, which when present is at concentrations ranging from about 0.3% to about 15%, by weight of the phase, more preferably at from about 0.5% to about 5% by weight of the phase. Suitable liquid crystalline phase inducing structurants include fatty acids (e.g. lauric acid, oleic acid, isostearic acid, linoleic acid) ester derivatives of fatty acids (e.g. propylene glycol isostearate, propylene glycol oleate, glyceryl isostearate) fatty alcohols, trihydroxystearin (available from Rheox, Inc. under the trade name THIXCIN® R). Preferably, the liquid crystalline phase inducing structurant is selected from lauric acid, trihydroxystearin, lauryl pyrrolidone, and tridecanol.

The multi-phase personal care compositions of the present invention can additionally comprise an organic cationic deposition polymer in the one or more phases as a deposition aid for the benefit agents described herein. Suitable cationic deposition polymers for use in the compositions of the present invention contain cationic nitrogen-containing moieties such as quaternary ammonium or cationic protonated amino moieties. The cationic protonated amines can be primary, secondary, or tertiary amines depending upon the particular species and the selected pH of the multi-phase personal care composition. Suitable cationic deposition polymers that would be useful in the compositions of the present invention are disclosed in the co-pending and commonly assigned U.S. Patent Application No. 60/628,036 filed on Nov. 15, 2003 by Wagner, et al titled “Depositable Solids.”

Nonlimiting examples of cationic deposition polymers for use in compositions include polysaccharide polymers, such as cationic cellulose derivatives. Preferred cationic cellulose polymers are the salts of hydroxyethyl cellulose reacted with trimethyl ammonium substituted epoxide, referred to in the industry (CTFA) as Polyquatemium 10 which are available from Amerchol Corp. (Edison, N.J., USA) in their Polymer KG, JR and LR series of polymers with the most preferred being KG-30M.

The multi-phase personal care composition of the present invention can comprise a particle. A water insoluble particle of various shapes and densities can be useful. In a preferred embodiment, the particle tends to have a spherical, an oval, an irregular, or any other shape in which the ratio of the largest dimension to the smallest dimension (defined as the Aspect Ratio) is less than about 10, preferably less than about 8, and still more preferably the Aspect Ratio of the particle is less than about 5. Preferably, the particle will also have physical properties which are not significantly affected by typical processing of the composition.

The multi-phase personal care composition of the present invention can comprise an exfoliant particle selected from the group consisting of polyethylene, microcrystalline wax, jojoba esters, amourphors silica, talc, tracalcium orthophosphate, and mixtures thereof. Exfoliant particles can be comprised in at least one phase of the multi-phase personal care composition at a level of less than about 10%, by weight of the composition.

The multi-phase personal care compositions of the present invention can comprise a shiny particle in at least one phase of the multi-phase personal care composition. Nonlimiting examples of shiny particles include the following: interference pigment, multi-layered pigment, metallic particle, solid and liquid crystals, and combinations thereof. An interference pigment is a pigment with pearl gloss prepared by coating the surface of a particle substrate material with a thin film. Interference pigments and hydrophobically modified interference pigments that are suitable for use in the compositions of the present invention are those disclosed in U.S. Pat. No. 6,395,691 issued to Liang Sheng Tsaur on May 28, 2002, U.S. Pat. No. 6,645,511 issued to Aronson, et al., U.S. Pat. No. 6,759,376 issued to Zhang, et al on Jul. 6, 2004, U.S. Pat. No. 6,780,826 issued on Aug. 24, 2004, U.S. Patent Application No. 2003/0054019 filed on May 21, 2002, published on Mar. 21, 2003 to Aronson, et al, as well as those pending and commonly assigned under U.S. Patent Application No. 60/469,570 filed on May 9, 2003 by Clapp, et al titled “Personal Care Compositions That Deposit Shiny Particles,” U.S. Patent Application No. 60/515,029 filed on Oct. 28, 2003, 2003 by Clapp, et al titled “Methods for Using Personal Care Compositions Containing Shiny Particles” and U.S. patent application Ser. No. 10/841,173 filed on May 7, 2004 by Clapp, et al titled “Personal Care Compositions Containing Hydrophobically Modified Interference Pigments.”

The multi-phase personal care composition of the present invention can comprise beads of any color and may be located in one or more phases of the of the multi-phase personal care composition. Suitable beads include those known in the art, including soft and hard beads. Suitable examples of soft beads include unispheres, made by Induchem, Unispheres NT-2806 (Pink). Suitable examples of hard beads include polyethylene or oxidized polyethylene, preferably those made by Accutech.

One or more of the phases of the multi-phase personal care composition can comprise a variety of additional optional ingredients. Such optional ingredients are most typically those materials approved for use in cosmetics and that are described in reference books such as the CTFA Cosmetic Ingredient Handbook, Second Edition. The Cosmetic, Toiletries, and Fragrance Association, Inc. 1988, 1992.

Other non limiting examples of these optional ingredients include vitamins and derivatives thereof (e.g., ascorbic acid, vitamin E, tocopheryl acetate, and the like), sunscreens; thickening agents (e.g., polyol alkoxy ester, available as Crothix from Croda), preservatives for maintaining the anti microbial integrity of the cleansing compositions, anti-acne medicaments (resorcinol, salicylic acid, and the like), antioxidants, skin soothing and healing agents such as aloe vera extract, allantoin and the like, chelators and sequestrants, skin lightening agents, and agents suitable for aesthetic purposes such as fragrances, essential oils, skin sensates, pigments, pearlescent agents (e.g., mica and titanium dioxide), lakes, colorings, and the like (e.g., clove oil, menthol, camphor, eucalyptus oil, and eugenol).

### Test Methods

Yield Stress and Zero Shear Viscosity Method: The Yield Stress and Zero Shear Viscosity of a phase of the present composition, can be measured either prior to combining in the composition, or after combining in the composition by separating the phase by suitable physical separation means, such as centrifugation, pipetting, cutting away mechanically, rinsing, filtering, or other separation means.

A controlled stress rheometer such as a TA Instruments AR2000 Rheometer is used to determine the Yield Stress and Zero Shear Viscosity. The determination is performed at 25° C. with the 4 cm diameter parallel plate measuring system and a 1 mm gap. The geometry has a shear stress factor of 79580 m⁻³ to convert torque obtained to stress.

First a sample of the phase is obtained and placed in position on the rheometer base plate, the measurement geometry (upper plate) moving into position 1 mm above the base plate. Excess phase at the geometry edge is removed by scraping after locking the geometry. If the phase comprises particles discernible to the eye or by feel (beads, e.g.) which are larger than about 150 microns in number average diameter, the gap setting between the base plate and upper plate is increased to the smaller of 4 mm or 8-fold the diameter of the 95^{th} volume percentile particle diameter. If a phase has any particle larger than 5 mm in any dimension, the particles are removed prior to the measurement.

The determination is performed via the programmed application of a continuous shear stress ramp from 0.1 Pa to 1,000 Pa over a time interval of 5 minutes using a logarithmic progression, i.e., measurement points evenly spaced on a logarithmic scale. Thirty (30) measurement points per decade of stress increase are obtained. Stress, strain and viscosity are recorded. If the measurement result is incomplete, for example if material flows from the gap, results obtained are evaluated and incomplete data points excluded. The Yield Stress is determined as follows. Stress (Pa) and strain (unitless) data are transformed by taking their logarithms (base 10). Log(stress) is graphed vs. log(strain) for only the data obtained between a stress of 0.2 Pa and 2.0 Pa, about 30 points. If the viscosity at a stress of 1 Pa is less than 500 Pa-sec but greater than 75 Pa-sec, then log(stress) is graphed vs. log(strain) for only the data between 0.2 Pa and 1.0 Pa, and the following mathematical procedure is followed. If the viscosity at a stress of 1 Pa is less than 75 Pa-sec, the zero shear viscosity is the median of the 4 highest viscosity values (i.e., individual points) obtained in the test, the yield stress is zero, and the following mathematical procedure is not used. The mathematical procedure is as follows. A straight line least squares regression is performed on the results using the logarithmically transformed data in the indicated stress region, an equation being obtained of the form:

Log(strain)=m*Log(stress)+b (1)

Using the regression obtained, for each stress value (i.e., individual point) in the determination between 0.1 and 1,000 Pa, a predicted value of log(strain) is obtained using the coefficients m and b obtained, and the actual stress, using Equation (1). From the predicted log(strain), a predicted strain at each stress is obtained by taking the antilog (i.e., 10^{x} for each x). The predicted strain is compared to the actual strain at each measurement point to obtain a % variation at each point, using Equation (2).

% variation=100*(measured strain−predicted strain)/measured strain (2)

The Yield Stress is the first stress (Pa) at which % variation exceeds 10% and subsequent (higher) stresses result in even greater variation than 10% due to the onset of flow or deformation of the structure. The Zero Shear Viscosity is obtained by taking a first median value of viscosity in Pascal-seconds (Pa-sec) for viscosity data obtained between and including 0.1 Pa and the Yield Stress. After taking the first median viscosity, all viscosity values greater than 5-fold the first median value and less than 0.2× the median value are excluded, and a second median viscosity value is obtained of the same viscosity data, excluding the indicated data points. The second median viscosity so obtained is the Zero Shear Viscosity.

Lather Volume Test: Lather volume of a cleansing phase, a surfactant component or a structured domain of a multi-phase personal care composition, is measured using a graduated cylinder and a rotating apparatus. A 1,000 ml graduated cylinder is used which is marked in 10 ml increments and has a height of 14.5 inches at the 1,000 ml mark from the inside of its base (for example, Pyrex No. 2982). Distilled water (100 grams at 25° C.) is added to the graduated cylinder. The cylinder is clamped in a rotating device, which clamps the cylinder with an axis of rotation that transects the center of the graduated cylinder. Inject 0.50 grams of a surfactant component or cleansing phase from a syringe (weigh to ensure proper dosing) into the graduated cylinder onto the side of the cylinder, above the water line, and cap the cylinder. When the sample is evaluated, use only 0.25 cc, keeping everything else the same. The cylinder is rotated for 20 complete revolutions at a rate of about 10 revolutions per 18 seconds, and stopped in a vertical position to complete the first rotation sequence. A timer is set to allow 15 seconds for lather generated to drain. After 15 seconds of such drainage, the first lather volume is measured to the nearest 10 ml mark by recording the lather height in ml up from the base (including any water that has drained to the bottom on top of which the lather is floating).

If the top surface of the lather is uneven, the lowest height at which it is possible to see halfway across the graduated cylinder is the first lather volume (ml). If the lather is so coarse that a single or only a few foam cells which comprise the lather (“bubbles”) reach across the entire cylinder, the height at which at least 10 foam cells are required to fill the space is the first lather volume, also in ml up from the base. Foam cells larger than one inch in any dimension, no matter where they occur, are designated as unfilled air instead of lather. Foam that collects on the top of the graduated cylinder but does not drain is also incorporated in the measurement if the foam on the top is in its own continuous layer, by adding the ml of foam collected there using a ruler to measure thickness of the layer, to the ml of foam measured up from the base. The maximum lather height is 1,000 ml (even if the total lather height exceeds the 1,000 ml mark on the graduated cylinder). 30 seconds after the first rotation is completed, a second rotation sequence is commenced which is identical in speed and duration to the first rotation sequence. The second lather volume is recorded in the same manner as the first, after the same 15 seconds of drainage time. A third sequence is completed and the third lather volume is measured in the same manner, with the same pause between each for drainage and taking the measurement.

The lather results after each sequence are added together and the Total Lather Volume determined as the sum of the three measurements, in milliters (“ml”). The Flash Lather Volume is the result after the first rotation sequence only, in ml, i.e., the first lather volume. Compositions according to the present invention perform significantly better in this test than similar compositions in conventional emulsion form.

Ultracentrifugation Method: The Ultracentrifugation Method is used to determine the percent of a structured domain or an opaque structured domain that is present in a multi-phase personal care composition that comprises a cleansing phase comprising a surfactant component. The method involves the separation of the composition by ultracentrifugation into separate but distinguishable layers. The multi-phase. personal care composition of the present invention can have multiple distinguishable layers, for example a non-structured surfactant layer, a structured surfactant layer, and a benefit layer.

First, dispense about 4 grams of multi-phase personal care composition into Beckman Centrifuge Tube (11×60 mm). Next, place the centrifuge tubes in an Ultracentrifuge (Beckman Model L8-M or equivalent) and ultracentrifuge using the following conditions: 50,000 rpm, 18 hours, and 25° C.

After ultracentrifuging for 18 hours, determine the relative phase volume by measuring the height of each layer visually using an Electronic Digital Caliper (within 0.01 mm). First, the total height is measured as Hₐ which includes all materials in the ultracentrifuge tube. Second, the height of the benefit layer is measured as H_{b}. Third, the structured surfactant layer is measured as H_{c}. The benefit layer is determined by its low moisture content (less than 10% water as measured by Karl Fischer Titration). It generally presents at the top of the centrifuge tube. The total surfactant layer height (Hₛ) can be calculated by this equation:

Hₛ=Hₐ−H_{b}

The structured surfactant layer components may comprise several layers or a single layer. Upon ultracentrifugation, there is generally an isotropic layer at the bottom or next to the bottom of the ultracentrifuge tube. This clear isotropic layer typically represents the non-structured micellar surfactant layer. The layers above the isotropic phase generally comprise higher surfactant concentration with higher ordered structures (such as liquid crystals). These structured layers are sometimes opaque to naked eyes, or translucent, or clear. There is generally a distinct phase boundary between the structured layer and the non-structured isotropic layer. The physical nature of the structured surfactant layers can be determined through microscopy under polarized light. The structured surfactant layers typically exhibit distinctive texture under polarized light. Another method for characterizing the structured surfactant layer is to use X-ray diffraction technique. Structured surfactant layer display multiple lines that are often associated primarily with the long spacings of the liquid crystal structure. There may be several structured layers present, so that H_{c} is the sum of the individual structured layers. If a coacervate phase or any type of polymer-surfactant phase is present, it is considered a structured phase.

Finally, the structured domain volume ratio is calculated as follows:

Structured Domain Volume Ratio=H_{c}/Hₛ*100%

If there is no benefit phase present, use the total height as the surfactant layer height, Hₛ=Hₐ.

### Method of Use

The multi-phase personal care compositions of the present invention are preferably applied topically to the desired area of the skin or hair in an amount sufficient to provide effective delivery of the skin cleansing agent, hydrophobic material, and particles to the applied surface. The compositions can be applied directly to the skin or indirectly via the use of a cleansing puff, washcloth, sponge or other implement. The compositions are preferably diluted with water prior to, during, or after topical application, and then subsequently the skin or hair rinsed or wiped off, preferably rinsed off of the applied surface using water or a water-insoluble substrate in combination with water. The present invention is therefore also directed to methods of cleansing the skin through the above-described application of the compositions of the present invention.

### Method of Manufacture

The multi-phase personal care compositions of the present invention may be prepared by any known or otherwise effective technique, suitable for making and formulating the desired multi-phase product form. It is effective to combine toothpaste-tube filling technology with a spinning stage design. Additionally, the present invention can be prepared by the method and apparatus as disclosed in U.S. Pat. No. 6,213,166 issued to Thibiant, et al. on Apr. 10, 2001. The method and apparatus allows two or more compositions to be filled in a spiral configuration into a single container using at least two nozzles which fill the container, which is placed on a static mixer and spun as the composition is introduced into the container.

Alternatively, the present invention can be prepared by a method disclosed in commonly owned U.S. patent application Ser. No. 10/837,214 Publication No. 2004/0219119 A1 entitled “Visually distinctive multiple liquid phase compositions” filed by Wei, et al. on Apr. 30, 2004, published on Nov. 18, 2004. The method and apparatus allows two separate compositions to be combined in predetermined amounts, blended into a single resultant composition with visually distinct phases, and filled by one nozzle into a single container that is lowered and rotated during filling.

If the multi-phase personal care compositions are patterned, it can be desirable to package these compositions in a transparent or translucent package such that the consumer can view the pattern through the package. Because of the viscosity of the subject compositions it may also be desirable to include instructions to the consumer to store the package upside down, on its cap to facilitate dispensing.

### EXAMPLES

The following examples described in Table 3 are non-limiting examples of the blooming perfume compositions:

TABLE 3
Blooming Perfume Compositions of the Present Invention

| | | Perfume | Perfume | Perfume | Perfume |
|---|---|---|---|---|---|
| | | 1 | 2 | 3 | 4 |
| Name | INCI Name | % wt. | % wt. | % wt. | % wt. |
| Beta | 2-Hexen-1-ol | 2.000 | 2.00 | 4.00 | 4.00 |
| Gamma | | | | | |
| Hexenol | | | | | |
| Cis 3 | (Z)-3-Hexen-1-ol | 3.000 | 3.00 | 7.00 | 7.00 |
| Hexenyl | acetate | | | | |
| Acetate | | | | | |
| Cyclo | Cyclo Galbanate | | | 2.00 | 2.00 |
| Galbanate | | | | | |
| Dihydro | 2,6-dimethyl-7- | 9.0 | | 14.00 | 12.00 |
| Myrcenol | Octen-2-ol | | | | |
| Ethyl | Ethyl Caproate | | | | 5.00 |
| Caproate | | | | | |
| Ethyl-2- | Butanoic acid, 2- | 5.000 | 5.00 | 10.00 | 7.00 |
| methyl | methyl-, ethyl | | | | |
| Butyrate | ester | | | | |
| Hexyl | Acetic acid, | | 5.0 | | 7.00 |
| Acetate | hexyl-ester | | | | |
| Melonal | 2,6-Dimethyl-5- | | 3.00 | 8.00 | 8.00 |
| | heptenal | | | | |
| Triplal | 2,4-Dimethyl-3- | 3.000 | 3.00 | 3.00 | 3.00 |
| | cyclohexene-1- | | | | |
| | carboxaldehyde | | | | |
| Anethol Usp | Benzene, 1- | 2.000 | 2.00 | 5.00 | 5.00 |
| | methoxy-4-(1- | | | | |
| | propenyl)- | | | | |
| Decyl | Decanal | 2.000 | 2.00 | 2.00 | 2.00 |
| Aldehyde | | | | | |
| Gamma | 2(3H)-Furanone, 5- | 4.000 | 4.00 | 3.00 | 3.00 |
| Decalactone | hexyldihydro- | | | | |
| Hexyl | Octanal, 2- | 15.00 | 15.00 | 5.00 | 5.00 |
| Cinnamic | (phenylmethylene)- | | | | |
| Aldehyde | | | | | |
| Hydroxycitr | Octanal, 7-hydroxy- | | 5.00 | 8.00 | 8.00 |
| onellal | 3,7-dimethyl- | | | | |
| Ionone | 3-Buten-2-one, 3- | 10.000 | 10.00 | 5.00 | 5.00 |
| Gamma | methyl-4-(2,6,6- | | | | |
| Methyl | trimethyl-2- | | | | |
| | cyclohexen-1-yl)- | | | | |
| Iso E Super | 7- | 20.00 | 20.00 | 10.00 | 8.0 |
| | acetyl,1,2,3,4,5,6,7, | | | | |
| | 8-octahydro-1,1,6, | | | | |
| | 7-tetramethyl | | | | |
| | naphthalene | | | | |
| Methyl | Cyclopentaneacetic | 20.00 | 16.00 | 14.00 | 9.00 |
| dihydrojasm | acid, 3-oxo-2- | | | | |
| onate | pentyl-, methyl ester | | | | |
| Phenoxy | Propanoic acid, 2- | 5.000 | 5.000 | | |
| Ethyl Iso | methyl-, 2- | | | | |
| Butyrate | phenoxyethyl ester | | | | |

The following examples described in Table 4 are non-limiting examples of lathering cleansing phase and non-lathering structured aqueous phase compositions of the present invention.

TABLE 4
Examples 1 and 2 of the Present Invention

| | Example. 1 | Example 2 |
|---|---|---|
| Ingredient | wt % | wt % |
| I. Lathering Cleansing Phase | | |
| Composition | | |
| Miracare SLB-365 (from Rhodia) | 47.4 | 47.4 |
| (Sodium Trideceth Sulfate, Sodium | | |
| Lauramphoacetate, Cocamide MEA) | | |
| Cocamide MEA | 3.0 | 3.0 |
| Guar Hydroxypropyltrimonium Chloride | 0.7 | 0.7 |
| (N-Hance 3196 from Aqualon) | | |
| PEG 90M (Polyox WSR 301 from Dow | 0.2 | 0.2 |
| Chemical) | | |
| Glycerin | 0.8 | 0.8 |
| Sodium Chloride | 3.5 | 3.5 |
| Disodium EDTA | 0.05 | 0.05 |
| Glydant | 0.67 | 0.67 |
| Citric Acid | 0.4 | 0.4 |
| Perfume 1 | 2.0 | |
| Perfume 3 | | 2.0 |
| Red 7 Ca Lake (From LCW) | 0.01 | 0.01 |
| Water | Q.S. | Q.S. |
| (pH) | (6.0) | (6.0) |
| II. Non-Lathering Structured Phase | | |
| Composition | | |
| Acrylates/Vinyl Isodecanoate | 1.0 | |
| Crosspolymer | | |
| (Stabylen 30 from 3 V) | | |
| Xanthan gum(Keltrol CGT from CP | 1.0 | |
| Kelco) | | |
| Petrolatum (Superwhite Protopet from | 10 | 75 |
| Witco) | | |
| Mineral Oil (Hydrobrite 1000PO from | | 25 |
| Crompton Corp.) | | |
| Triethanolamine | 1.5 | |
| Sodium Chloride | 3.5 | |
| Glydant | 0.37 | |
| Water and Minors | Q.S. | |
| (pH) | (6.0) | N/A |

### Method of Making Example 1-2

The compositions described above can be prepared by conventional formulation and mixing techniques. The lathering cleansing phase composition can be prepared by forming the following premixes: adding citric acid into water at 1:1 ratio to form a citric acid premix, add polyox WSR-301 into glycerin at 1:3 ratio to form a polyox-glycerin premix, and add cosmetic pigment into glycerin at 1:20 ratio to form a pigment-glycerin premix and mix well using a high shear mixer. Then, add the following ingredient in the main mixing vessel in the following sequence: water, polyox premix, citric acid premix, disodium EDTA, and Miracare SLB-365. Mix for 30 mins, then begin heating the batch to 120 F. Add CMEA and mix until homogeneous. Then, cool the batch to ambient temperature and add the following ingredients: sodium chloride, glydant, cosmetic pigment premix and perfume. Mix the batch for 60 mins. Check pH and adjust pH using citric acid or caustic solution if needed.

The non-lathering structured phase (Ex 1) can be prepared by slowly adding Stabylene 30 into water with continuous mixing. Then, add Keltrol CG-T. Heat the batch to 85 C with continuous agitation. Then, add Superwhite Protopet. Cool down the batch to ambient temperature. Then, add Triethanolamine. The batch becomes viscous. Add sodium chloride, glydant and mix until homogeneous.

The non-lathering structured phase (Ex 2) can be prepared by adding petrolatum into a mixing vessel. Heat the vessel to 88 C. Then add mineral oil with agitation. Once homogenous, allow the vessel to cool down with slow agitation.

The lathering cleansing and non-lathering structured aqueous phases can be combined by first placing the separate phases in separate storage tanks having a pump and a hose attached. The phases are then pumped in predetermined amounts into a single combining section. Next, the phases are moved from the combining sections into the blending sections and the phases are mixed in the blending section such that the single resulting product exhibits a distinct pattern of the phases. The pattern is selected from the group consisting of striped, marbled, geometric, and mixtures thereof. The next step involves pumping the product that was mixed in the blending section via a hose into a single nozzle, then placing the nozzle into a container and filing the container with the resulting product. The stripe size is about 6 mm in width and 100 mm in length.

The following examples described in Table 5 are non-limiting examples of the multi-phase personal care composition of the present invention, which is an in-shower body lotion product.

TABLE 5
Examples of in-shower body lotion

Example

| | |
|---|---|
| Ex. 3 | Ex. 4 |

FIRST PHASE

| | Amount (By | Amount (By |
|---|---|---|
| | Weight of | Weight of |
| Ingredients | First Phase) | First Phase) |
| Petrolatum^{a} | 22.0% | 22.0% |
| Diisopropyl Sebacate^{b} | 3.5% | 3.5% |
| Hydroxypropyl Starch | 3.5% | 3.5% |
| Phosphate^{c} | | |
| Stearyl Alcohol, Cetyl | 2.4% | 2.4% |
| Alcohol, and Polysorbate | | |
| 60 blend^{d} | | |
| Perfume 2 | 1.2% | |
| Preservative^{e} | 0.293% | 0.293% |
| Phenoxyethanol | 0.25% | 0.25% |
| Disodium EDTA^{f} | 0.12% | 0.12% |
| Water | Balance to 100% | Balance to 100% |

SECOND PHASE

| | Amount (By | Amount (By |
|---|---|---|
| | Weight of | Weight of |
| Ingredients | Second Phase) | Second Phase) |
| Petrolatum^{a} | 22.0% | 22.0% |
| Colorant^{g} | 0.003% | 0.003% |
| Diisopropyl Sebacate^{b} | 3.5% | 3.5% |
| Hydroxypropyl Starch | 3.5% | 3.5% |
| Phosphate^{c} | | |
| Stearyl Alcohol, Cetyl | 2.4% | 2.4% |
| Alcohol, and Polysorbate | | |
| 60 blend^{d} | | |
| Perfume 4 | | 1.2% |
| Preservative^{e} | 0.293% | 0.293% |
| Phenoxyethanol | 0.25% | 0.25% |
| Disodium EDTA^{f} | 0.12% | 0.12% |
| Water | Balance to 100% | Balance to 100% |

^{a}Commercially available from Crompton Witco under the tradename G-2180 Petrolatum;
^{b}Commercially available from Noveon under the tradename SCHERCEMOL DIS.
^{c}Commercially available from National Starch under the tradename STRUCTURE XL.
^{d}Commercially available from Croda under the tradename POLAWAX Pastilles.
^{e}Commercially available from Lonza under the tradename GLYDANT PLUS Liquid.
^{f}Commercially available from Akzo Nobel under the tradename DISSOLVINE NA2-S.
^{g}Commercially available under the tradename D&C Violet 2.

### Method of Making Examples 3 and 4

The first and second phases of the multi-phase personal care composition exemplified above are both opaque. The viscosity of the first phase of the in-shower body lotion is about 8,500 Pa·s. The viscosity of the second phase of the in-shower body lotion is about 8,000 Pa·s. The first and second phases are both oil-in-water emulsions and are both non-Newtonian. The first and second phases are combined as described below and form a visually distinct striped pattern.

The multi-phase personal care composition exemplified above, which is an in-shower body lotion, is made by separately making the first phase and the second phase, and then combining them according to the process described in US 2004/0219119 A1 (Case 9218) to form the finished multi-phase personal care composition.

The first phase is made according to the following procedure. Add about 300 grams of water to a first beaker and heat the water to about 85-90° C. In a second beaker, add about 66 grams of melted petrolatum and heat to about 85-90° C. Add about 7.2 grams of POLAWAX to the second beaker and mix. Add about 10.5 grams of STRUCTURE XL to the second beaker and mix. Take about 199.892 grams of heated water from the first beaker, add it to the second beaker, and mix. Add about 0.36 grams of Disodium EDTA to the second beaker. Add about 0.75 grams of Phenoxyethanol to the second beaker and mix. Move the second beaker to a water bath, continue mixing, and adjust the temperature of the contents of the second beaker to about 47° C. Add about 10.5 grams of SCHERCEMOL DIS to the second beaker at about 47° C. Add about 1.198 grams of GLYDANT PLUS liquid to the second beaker at about 46° C. and mix. Add about 3.6 grams of perfume to the second beaker at about 45° C. and mix. Cool the contents of the second beaker while mixing and then empty the contents into a first storage tank.

The second phase is made according to the following procedure. Add about 300 grams of water to a third beaker and heat the water to about 85-90° C. In a fourth beaker, add about 66 grams of melted petrolatum and heat to about 85-90° C. Add about 0.008 grams of colorant to the fourth beaker and mix until the colorant is dissolved in the petrolatum. Add about 7.2 grams of POLAWAX to the fourth beaker and mix. Add about 10.5 grams of STRUCTURE XL to the fourth beaker and mix. Take about 199.884 grams of heated water from the third beaker, add it to the fourth beaker, and mix. Add about 0.36 grams of disodium EDTA to the fourth beaker. Add about 0.75 grams of phenoxyethanol to the fourth beaker and mix. Move the fourth beaker to a water bath, continue mixing, and adjust the temperature of the contents of the fourth beaker to about 47° C. Add about 10.5 grams of SCHERCEMOL DIS to the fourth beaker at about 47° C. Add about 1.198 grams of GLYDANT PLUS liquid to the fourth beaker at about 46° C. and mix. Add about 3.6 grams of perfume to the fourth beaker at about 45° C. and mix. Cool the contents of the fourth beaker while mixing and then empty the contents into a second storage tank.

The first and second phases combined to form a multi-phase personal care composition according to a process similar to that described in U.S. patent application Ser. No. 10/837,214 Publication No. 2004/0219119 A1 entitled “Visually distinctive multiple liquid phase compositions” filed by Wei, et al. on Apr. 30, 2004, published on Nov. 18, 2004, except that a static mixer is not utilized. The first phase is pumped from the first storage tank into a receiving cavity. The second phase is pumped from the second storage tank into the same receiving cavity. The first and second phases are then pumped out of the receiving cavity and through a filling nozzle to form the multi-phase personal care composition. A plastic bottle, or other package, is placed directly underneath the filling nozzle to receive the multi-phase personal care composition from the filling nozzle. The plastic bottle is positioned on a bottle holding stand that lowers and rotates the bottle during filling. As the multi-phase personal care composition flows from the filling nozzle, the bottle holding stand lowers and rotates the bottle during filling at about 250 rpm. When the bottle is filled with the multi-phase personal care composition, the process is complete. The phases in the multi-phase personal care composition form a visually distinct pattern.

The following examples described in Table 6 are non-limiting examples 5-7 of the multi-phase personal care composition of the present invention, which is a cleansing and conditioning product.

TABLE 6
Examples of multi-phase - cleansing and conditioning

| | Ex. 5 | Ex. 6 | Ex. 7 |
|---|---|---|---|
| Ingredient | wt % | wt % | wt % |
| Cleansing Phase Composition | | | |
| Ammonium Laureth-3 Sulfate | 3.0 | 3.0 | 3.0 |
| Sodium Lauroamphoacetate | 16.7 | 16.7 | 16.7 |
| (Miranol L-32 Ultra from Rhodia) | | | |
| Surfactant Blend (Miracare SLB-365 from Rhodia) | — | — | — |
| Ammonium Lauryl Sulfate | 1.0 | 1.0 | 1.0 |
| Ammonium Laureth Sulfate | | | |
| Lauric Acid (Emry 625) | 0.9 | 0.9 | 0.9 |
| Trihydroxystearin (Thixcin R) | 2.0 | 2.0 | 2.0 |
| Guar Hydroxypropyltrimonium Chloride | 0.17 | 0.75 | 0.75 |
| (N-Hance 3196 from Aqualon) | | | |
| Guar Hydroxypropyltrimonium Chloride (Jaguar | 0.58 | — | — |
| C-17 from Rhodia) | | | |
| Polyquaterium 10 | 0.45 | — | — |
| (UCARE polymer JR-30M from Amerchol) | | | |
| Polymethacrylamidopropyltrimonium Chloride | — | 0.24 | — |
| (Polycare 133 from Rhodia) | | | |
| Polyquaternium-39 | — | 0.81 | — |
| (Merqurt Plus 3300 from Calgon) | | | |
| PEG 90M (Polyox WSR 301 from Union Carbide) | 0.25 | — | — |
| PEG-14M (Polyox WSR N-3000 H from Union | 0.45 | 2.45 | 2.45 |
| Carbide) | | | |
| Linoleamidoprypyl PG-Dimonium Chloride | — | 1.0 | 4.0 |
| Phosphate Dimethicone | | | |
| (Monasil PLN from Uniqema) | | | |
| Dimethicone (Viscasil 330M from General | — | — | — |
| Electric) | | | |
| Ethylene Glycol Distearate | | | |
| Glycerin | 1.4 | 4.9 | 4.9 |
| Sodium Chloride | 0.3 | 0.3 | 0.3 |
| Sodium Benzoate | 0.25 | 0.25 | 0.25 |
| Disodium EDTA (Hampene NA2/Dissolvine NA- | 0.13 | 0.13 | 0.13 |
| 2X) | | | |
| Glydant | 0.37 | 0.37 | 0.37 |
| DMDM Hydantoin (Lonza) | — | — | — |
| D&C Red#30 Talc Lake | — | — | — |
| Citric Acid | 1.6 | 0.95 | 0.95 |
| Titanium Dioxide | 0.5 | 0.5 | 0.5 |
| Perfume 1 | 1.0 | | |
| Perfume 3 | | | 1.0 |
| Perfume 2 | | 1 | |
| Water | Q.S. | Q.S. | Q.S. |
| Expancel 091-DE-40-D30 (Expancel Corp.) | 0.00001 | 0.00001 | 0.00001 |
| Benefit Phase Composition | | | |
| Stearamidopropyldimethylamine (1) | 2.00 | 1.60 | 2.00 |
| Stearamidoethyldiethylamine (2) | | | |
| Behentrimonium chloride (3) | — | 3.4 | — |
| L-Glutamic Acid (4) | 0.64 | 0.51 | 0.64 |
| Cetyl Alcohol (5) | 2.50 | 2.32 | 3.75 |
| Stearyl Alcohol (6) | 4.50 | 4.2 | 6.75 |
| Oleyl Alcohol (7) | — | — | — |
| Mineral Oil (8) | | — | — |
| Dimethicone Blend (9) | — | 4.2 | |
| Silicone Emulsion (10) | | | |
| Dimethicone silicone fluid blend (11) | 4.2 | — | 4.2 |
| Benzyl Alcohol | 0.40 | 0.40 | 0.40 |
| EDTA | 0.10 | 0.13 | 0.10 |
| Kathon CG (12) | 0.03 | 0.03 | 0.03 |
| Methyl Paraben | | | |
| Propyl Paraben | | | |
| Panthenyl Ethyl Ether | 0.05 | 0.1 | |
| Panthenol | 0.09 | 0.09 | |
| Sodium Chloride | — | 0.01 | — |
| Water | qs | qs | qs |
| Ratio Cleansing Phase/Benefit Phase | 60/40 | 70/30 | 70/30 |

(1) Stearamidopropyldimethylamine: AMIDOAMINE MPS obtained from Nikko;
(2) Stearamidoethyldiethylamine: AMIDOAMINE S obtained from Nikko;
(3) Behentrimonium chloride available from Clariant as Genamin KDMP;
(4) L-glutamic acid: L-GLUTAMIC ACID (cosmetic grade) obtained from Ajinomoto;
(5) Cetyl Alcohol: KONOL series obtained from New Japan Chemical;
(6) Stearyl Alcohol: KONOL series obtained from New Japan Chemical;
(7) Oleyl Alcohol: UNJECOL 90BHR obtained from New Japan Chemical;
(8) Mineral Oil: BENOL obtained from Witco;
(9) A 60% 350 cst and 40% 18,000,000 cst dimethicone fluid blend available from General Electric Silicones Products;
(10) Dow Cornining HMW 2220 Non-ionic emulsion;
(11) Dimethicone fluid blend (0.5 MM cSt/200 cSt [15/85 v/v %]) available from General Electric Silicones Products; and
(12) Kathon CG: Mixture of methylcholorisothiazoline and methylisothiazoline obtained from Rohm & Hass Co.

Prepare cleansing phase composition of example 5 by first creating the following premixes: citric acid in water premix at 1:3 ratio, Guar polymer premix with Jaguar C-17 and N-Hance 3196 in water at about 1:10 ratio, UCARE premix with JR-30 M in water at about 1:30 ratio, and Polyox premix with PEG-90M and PEG-14M in Glycerin at about 1:2 ratio. Then, add the following ingredients into the main mixing vessel: ammonium lauryl sulfate, ammonium laureth-3 sulfate, citric acid premix, Miranol L-32 ultra, sodium chloride, sodium benzoate, disodium EDTA, lauric acid, Thixcin R, Guar premix, UCARE premix, Polyox Premix, and the rest of water. Then, heat the vessel with agitation until it reaches 190° F. (88° C.). Let it mix for about 10 minutes. Cool the batch with a cold water bath with slow agitation until it reaches 110° F. (43° C.). Add the following ingredients: Glydant, perfume, Titanium Dioxide. Mix until a homogeneous solution forms.

Prepare example 6 of cleansing phase composition by first creating the following premixes: citric acid in water premix at about 1:3 ratio, Guar polymer premix with N-Hance 3196 in water at about 1:10 ratio, and Polyox premix with PEG-14M in Glycerin at about 1:2 ratio. Then, add the following ingredients into the main mixing vessel: ammonium lauryl sulfate, ammonium laureth-3 sulfate, citric acid premix, Miranol L-32 ultra, sodium chloride, sodium benzoate, disodium EDTA, lauric acid, Thixcin R, Guar premix, Polyox Premix, Polycare 133, Merquat Plus 3300, Monosil PLN, and the rest of water. Then, heat the vessel with agitation until it reaches 190° F. (88° C.). Mix for about 10 minutes. Next, cool the batch with a cold water bath with slow agitation until it reaches 110° F. (43° C.). Finally, add the following ingredients: Glydant, perfume, Titanium Dioxide. Mix until a homogeneous solution forms.

Prepare examples 7 of cleansing phase by first creating the following premixes: citric acid in water premix at about 1:3 ratio, Guar polymer premix with N-Hance 3196 in water at about 1:10 ratio, and Polyox premix with PEG-14M in Glycerin at about 1:2 ratio. Then, add the following ingredients into the main mixing vessel: ammonium lauryl sulfate, ammonium laureth-3 sulfate, citric acid premix, Miranol L-32 ultra, sodium chloride, sodium benzoate, disodium EDTA, lauric acid, Thixcin R, Guar premix, Polyox Premix, Monasil PLN, and the rest of water. Then, heat the vessel with agitation until it reaches 190° F. (88° C.). Mix the vessel for about 10 minutes. Next, cool the batch with a cold water bath with slow agitation until it reaches 110° F. (43° C.). Finally, add the following ingredients: Glydant, perfume, Titanium Dioxide. Mix until a homogeneous solution forms.

For preparing benefit phase compositions of examples 5 through 7, mix water, stearamidopropyldimethylamine and about 50% of L-glutamic acid at a temperature above 70° C. Then, add the high melting point fatty compounds and benzyl alcohol with agitation. Cool down below 60° C., then add the remaining L-glutamic acid and other remaining components with agitation, then cool down to about 30° C.

The following examples described in Table 7 are non-limiting examples 8 and 9 of the multi-phase personal care composition of the present invention, which are a shampoo product.

TABLE 7
Multi-phase Shampoo Examples of the Present Invention

| | Ex. 8 | Ex. 9 |
|---|---|---|
| Cleansing Phase Composition | | |
| Ammonium Laureth-3 Sulfate | 12 | 10 |
| Ammonium Lauryl Sulfate | 2 | 6 |
| Cocamidopropyl Betaine | 2 | |
| Coconutmonoethanol amide (CMEA, Mona Industries) | 2 | 0.8 |
| Cetyl alcohol | | 0.6 |
| Ethylene Glycol Distearate (EGDS) | | 1.5 |
| Structure Plus (National Starch) | | 3 |
| Carbopol Aqua SF-1 (30%) (Noveon) | 3 | |
| Polyquaterium 10, (UCARE polymer JR-30M from | 0.25 | |
| Amerchol) | | |
| Polymethacrylamidopropyltrimonium Chloride | | 0.13 |
| (Polycare 133 from Rhodia) | | |
| Dow Corning 1870 (silicone nanoemulsion) | 2 | |
| Puresyn 6 (1-decene homopolymer) | | 0.3 |
| Kathon CG (Rhom & Haas) | 0.0005 | 0.0005 |
| Disodium EDTA (Dissolvine NA-2S, Akzo Nobel) | 0.1274 | 0.1274 |
| Sodium chloride (Morton) | 0.5 | 0.7 |
| Sodium Citrate Dihydrate | 0.4 | 0.4 |
| Citric Acid (Hoffman-Laroche) | 0.15 | 0.15 |
| Perfume 1 | 2.0 | |
| Perfume 3 | | 1.5 |
| Water | q.s. | q.s. |
| Benefit Phase Compositions | | |
| Ammonium Laureth-3 Sulfate | 12 | 10 |
| Ammonium Lauryl Sulfate | 2 | 6 |
| Cocamidopropyl Betaine (30%) (Goldschmidt | 2 | |
| Chemical) | | |
| Coconutmonoethanol amide (Mona Industries) | 2 | 0.8 |
| Ethylene Glycol Distearate (EGDS) | | 1.5 |
| Cetyl Alcohol | | 0.6 |
| Structure Plus (National Starch) | | 3 |
| Carbopol Aqua SF-1 (30%) (Noveon) | 3 | |
| Polyquaterium 10, (UCARE polymer JR-30M from | 0.25 | |
| Amerchol) | | |
| Polymethacrylamidopropyltrimonium Chloride | | 0.13 |
| (Polycare 133 from Rhodia) | | |
| Dimethicone (Viscasil 330M from General Electric) | | 2 |
| Dow Corning 1664 (silicone microemulsion) | 2 | |
| Puresyn 6 (1-decene homopolymer) | | 0.3 |
| Kathon CG (Rhom & Haas) | 0.0005 | 0.0005 |
| Disodium EDTA (Dissolvine NA-2S, Akzo Nobel) | 0.1274 | 0.1274 |
| Sodium Citrate Dihydrate | 0.4 | 0.4 |
| Citric Acid (Hoffman-Laroche) | 0.15 | 0.15 |
| FD&C Blue # 1 Aluminum Lake (Sun Chem.) | .003 | .002 |
| D&C Red # 7 Ca Lake (Sun Chem.) | | .01 |
| Perfume | 0.6 | 0.6 |
| Water | qs | Qs |
| Ratio Cleansing Phase/Benefit Phase | 90/10 | 70/30 |

### Cleansing Phase/Benefit Phase Compositions:

In an appropriate vessel, add distilled water and stir at an appropriate speed (100-200 ppm) using an appropriate sized stir blade. If needed, add the anionic polymer (Carbopol Aqua SF-1), cationic polymers (Polyquatemium-10, Polycare 133) and stir briefly and slowly to wet and disperse the polymer. While continuing to stir, if needed, add the citiric acid solution (50%) drop wise to the mix vessel to reduce pH until solution becomes clear. Add surfactants (ALS, AE3S, CAPB,) to the mixture. Heat the mixture to 60° C. and while stirring add CMEA, EGDS, and Cetyl alcohol to the mixture. Mix until homogeneous. Cool the solution to room temperature while stirring and add Silicone(s), Puresyn, Kathon, EDTA, Mackstat DM-C, D&C pigment, and perfume. Finally, adjust pH of the product within the preferred specified range of from about 5.5 to about 6.5.

Match the densities of the cleansing and benefit phases within 0.05 g/cm³. Combine these phases by first placing the separate phases in separate storage tanks having a pump and a hose attached. Then, pump the phases in predetermined amounts into a single combining section. Next, move the phases from the combining sections into blending sections and mix the phases in the blending section such that the single resulting product exhibits a distinct pattern of phases. Next, pump the product that was mixed in the blending section via a hose into a single nozzle into a spinning container, and fill the container from the bottom to the top with the resulting product.

The following examples described in Table 8 are non-limiting examples 10 and 11 of the multi-phase personal care composition of the present invention, which are a conditioner product.

TABLE 8
Multi-phase Conditioner Examples of the Present Invention

| | Example 10 | Example 11 |
|---|---|---|
| Conditioning Phase Composition | | |
| Stearamidopropyldimethylamine (1) | 2.0 | 1.2 |
| L-Glutamic acid (2) | 0.64 | 0.38 |
| Quaternium-18 (21) | — | 0.5 |
| Cetyl alcohol (3) | 2.5 | 2.00 |
| Stearyl alcohol (4) | 4.5 | 3.60 |
| Dimethicone blend (5) | — | 1.5 |
| Dimethicone/Cyclomethicone blend (6) | 4.2 | — |
| Benzyl alcohol (7) | 0.4 | 0.4 |
| EDTA (8) | 0.1 | 0.1 |
| Disodium EDTA (19) | — | — |
| Kathon CG (9) | 0.03 | 0.03 |
| Panthenyl Ethyl Ether (10) | 0.05 | 0.06 |
| Panthenol (11) | 0.09 | 0.05 |
| Perfume | 0.25 | 0.30 |
| Deionized Water | Qs | Qs |
| Benefit Phase Composition | | |
| Behetrimonium Chloride (13) | 2.25 | 3.38 |
| Cetyl alcohol | 1.86 | 2.32 |
| Stearyl alcohol | 4.64 | 4.18 |
| Dimethicone/Cyclomethicone blend (6) | — | 4.2 |
| Aminosilicone (15) | 3.5 | — |
| C13–C16 Isoparaffin (16) | 1.5 | — |
| Benzyl alcohol | 0.4 | 0.4 |
| Disodium EDTA (19) | 0.13 | 0.13 |
| EDTA (8) | — | — |
| Kathon CG | 0.033 | 0.033 |
| Panthenyl Ethyl Ether | 0.05 | 0.05 |
| Panthenol | 0.05 | 0.05 |
| Sodium hydroxide | 0.014 | 0.014 |
| Isopropyl alcohol | 0.9 | 0.9 |
| Pigment (17) | 0.08 | 0.08 |
| Perfume | 0.5 | 0.5 |
| Deionized Water | Qs | qs |
| Ratio Conditioning Phase/Benefit Phase | 20/80 | 20/80 |

(1) supplied by Inolex under trade name Lexamine S-13;
(2) supplied by Ajinomoto;
(3) supplied by Procter & Gamble;
(4) supplied by Procter & Gamble;
(5) supplied by GE Silicones as a blend of dimethicone having a viscosity of 18,000,000 mPa · s and dimethicone having a viscosity if 200 mPa · s;
(6) supplied by GE Silicone as a blend of dimethicone having a viscosity if 18,000,000 mPa · s and cyclopentasiloxane;
(7) supplied by Haarman & Reimer;
(8) supplied by BASF as Ethylene Diamine Tetracetic Acid;
(9) supplied by Rohm & Haas;
(10) supplied by Roche;
(11) supplied by Roche;
(13) supplied by Clariant;
(15) supplied by GE Silicones as reference number Y-14900;
(16) supplied by Nisseki as Isosol 400;
(17) supplied by Rona;
(18) supplied by Clariant as Genamin KDMP;
(19) supplied by SCAL;
(20) supplied by Croda as IncromineBB; and
(21) supplied by Goldschmidt.

In the conditioning phase compositions of examples 10 and 11, mix water, stearamidopropyldimethylamine, and L-glutamic acid at a temperature above 70° C. Then, add cetyl alcohol, stearyl alcohol, and benzyl alcohol with agitation. Cool down below 60° C., then add silicones, kathon, EDTA, panthenyl ethyl ether, panthenol and perfume with agitation. Then, cool down to about 30° C. In the benefit phase compositions of examples 10 and 11, water and benetrimonium chloride at a temperature above 70° C. Then, add cetyl alcohol, stearyl alcohol, and benzyl alcohol with agitation. Cool down below 60° C., then add amino-silicones, kathon, EDTA, panthenyl ethyl ether, panthenol, coloring pigment and perfume with agitation. Then, cool down to about 30° C.

Match the densities of the conditioning and benefit phases within 0.05 g/cm³. Combine these phases by first placing the separate phases in separate storage tanks having a pump and a hose attached. Then, pump the phases in predetermined amounts into a single combining section. Next, move the phases from the combining sections into blending sections and mix the phases in the blending section such that the single resulting product exhibits a distinct pattern of phases. Select the pattern from the group consisting of striped, marbled, geometric, and mixtures thereof. Next, pump the product that was mixed in the blending section via a hose into a single nozzle into a spinning container, and fill the container from the bottom to the top with the resulting product.

All parts, ratios, and percentages herein, in the Specification, Examples, and Claims, are by weight and all numerical limits are used with the normal degree of accuracy afforded by the art, unless otherwise specified.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A multi-phase, personal care composition comprising: a first phase and a second phase; said multi-phase personal care composition comprising at least 0.25%, by weight of said multi-phase personal care composition, of blooming perfume ingredients having Kovat's Index of less than about 1500.

2. The multi-phase, personal care composition of claim 1, wherein said blooming perfume ingredients have a boiling point of less than about 260° C. and a ClogP of about 1.5 to about 4.0.

3. The multi-phase, personal care composition of claim 1, wherein said multi-phase personal care composition further comprises non-blooming perfume ingredients having a Kovat's Index of greater than 1500.

4. The multi-phase, personal care composition of claim 1, where said non-blooming perfume ingredients have a boiling point of about 260° C. or higher, and a ClogP of higher than about 2.5.

5. The multi-phase, personal care composition of claim 3, further comprises a total perfume composition comprises said blooming perfume ingredients and said non-blooming ingredients, wherein the weight percentage of blooming perfume ingredients comprises at least 10% by weight of the total perfume composition.

6. The multi-phase, personal care composition of claim 1, wherein said multi-phase personal care composition comprises at least 0.30%, by weight of said multi-phase personal care composition, of blooming perfume ingredients having Kovat's Index of less than about 1500.

7. The multi-phase, personal care composition of claim 1, wherein said multi-phase personal care composition comprises at least 0.40%, by weight of multi-phase personal care composition, of blooming perfume ingredients having Kovat's Index of less than about 1500.

8. The multi-phase, personal care composition of claim 1, wherein said first phase is a cleansing phase comprising from about 2% to about 23.7%, by weight of said first phase, of said surfactant component.

9. The multi-phase, personal care composition of claim 8, wherein said first phase comprises said blooming perfume ingredients.

10. The multi-phase, personal care composition of claim 8, wherein said cleansing phase provides a Yield Stress of greater than about 1.5 Pascal.

11. The multi-phase, personal care composition of claim 1, wherein said personal care composition is a bar soap.

12. The multi-phase personal care composition of claim 1, wherein said personal care composition is a body wash.

13. The multi-phase, personal care composition of claim 1, wherein said personal care composition is a shampoo.

14. The multi-phase, personal care composition of claim 1, wherein said personal care composition is conditioner.

15. The multi-phase, personal care composition of claim 1, wherein said personal care composition is in-shower body moisturizer.

16. The multi-phase, personal care composition of claim 1, wherein said second phase is selected from the group consisting of a cleansing phase, a benefit phase, a non-lathering structured aqueous phase, and combinations thereof.

17. The multi-phase, personal care composition of claim 1, wherein said second phase is a benefit phase comprises hydrophobic material with a Vaughan Solubility Parameter of from about 5 to about 15.

18. The multi-phase, personal care composition of claim 1, wherein said second phase has a Consistency Value (K) of from about 30 to about 350 Pa-s.

19. The multi-phase, personal care composition of claim 1, wherein said first phase is visually distinct from said second visually distinct phase.

20. The multi-phase, personal care composition of claim 19 wherein said first phase and second phase form a pattern.

21. The multi-phase, personal care composition of claim 19, wherein said pattern is selected from the group consisting of striped, geometric, marbled, and combinations thereof.

22. The multi-phase, personal care composition of claim 19, wherein said composition is packaged in a container such that said pattern is visible through said container.

23. The multi-phase, personal care composition of claim 1, wherein said first phase is a cleansing phase comprising surfactant and water.

24. The multi-phase, personal care composition of claim 1, wherein said cleansing phase further comprises:
(i) at least one electrolyte;
(ii) at least one alkanolamide; and
(iii) water;
wherein said cleansing phase is non-Newtonian shear thinning; and wherein said cleansing phase has a viscosity of equal to or greater than about 3000 cps.

25. The multi-phase, personal care composition of claim 1, wherein said surfactant component further comprises:
i. at least one nonionic surfactant having an HLB from about 3.4 to about 15.0; and
ii. at least one amphoteric surfactant; wherein said composition further comprises an electrolyte.

26. The multi-phase, personal cleansing composition of claim 1, wherein said composition further comprises a benefit component, wherein said benefit component are selected from the group consisting of emollients, particles, beads, skin whitening agents, fragrances, colorants, vitamins and derivatives thereof, sunscreens, preservatives, anti-acne medicaments, antioxidant, chelators, essential oils, skin sensates, antimicrobial, and mixtures thereof.

27. The multi-phase personal care composition of claim 1, wherein said first phase and second phase are blended.
